(19) United States
(12) Patent Application Publication
(10) Pub. No.: US 2002/0099032 A1
(43) Pub. Date: Jul. 25, 2002

(54) Preparations and method of producing the same

(76) Inventors: Kiyotsugu Higashi, Osaka-shi (JP); Chikara Miura, Osaka-shi (JP); Kentaro Iida, Osaka-shi (JP); Yukiko Onaka, Osaka-shi (JP); Tomoharu Nishimori, Osaka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 09/986,442

(22) Filed: Nov. 8, 2001

(30) Foreign Application Priority Data
Nov. 10, 2000 (JP) 344317/2000
Nov. 10, 2000 (JP) 344315/2000

(51) Int. Cl. A61K 31/7008; A61K 31/51

(52) U.S. Cl. 514/62; 514/276

(57) ABSTRACT

Incorporation of an aminosugar (e.g., glucosamine) to a preparation make a vitamin B1 stable. The content of the aminosugar is an effective amount to stabilize the vitamin B1, and is, for example, not less than 0.1 part by weight relative to 1 part by weight of the vitamin B1. Incorporation of the aminosugar can improve the disintegrativity of a solid preparation comprising a glycosaminoglycan (a hyaluronic acid, a chondroitin or a salt thereof). The content of aminosugars is not less than 0.1 part by weight relative to 1 part by weight of glycosaminoglycans. The solid preparation can inhibit forming gel masses of glycosaminoglycan and can improve the disintegrativity. Moreover, a joint disorder such as arthralgia can be improved by combination of the vitamin B1 and the glucosamine (e.g., glucosamine or a salt thereof).

### FIELD OF THE INVENTION

This invention relates to a preparation in which a vitamin B1 is stabilized, particularly a preparation (particularly, a solid preparation) improved in disintegrativity (disintegration properties) as well as stabilization of the vitamin B1. This invention further relates to a composition for preventing or treating a joint disorder, which comprises a vitamin B1 and an aminosugar (especially, a glucosamine), if necessary, a glycosaminoglycan.

### BACKGROUND OF THE INVENTION

Many pharmaceutical preparations comprising vitamin B1 are commercially available. Concretely, a vitamin B1 is utilized for Wernicke's encephalopathy, peripheral neuropathy (peripheral nervous system disorder), central neuropathy (central nervous system disorder), neuralgia, myalgia, arthralgia (lumbagia, stiff shoulder, frozen shoulder), numbness of hands and feet, therapy of asthenopia, constipation, nutrition, etc. A vitamin B1 is effective in arthralgia, however effective in relatively slight symptom of arthralgia.

The joints (of human and animals) are constantly subjected to stress and strain from mechanical forces that can result in a joint disorder such as arthralgia, arthritis, osteoarthritis, and stiffness. The causes of a joint disorder include various kinds such as a joint deformation, bacterium infection (microbism), virus infection, injury, immuno-disease such as allergy, rheumatism and dysbolism caused by nephritis. An inflammatory disorder such as arthriris is caused so that a patient suffers from arthralgia and joint stiffness can not maintain normal joint flexibility and mobility, and comfortable joint movement is disturbed by a joint pain and stiffness. Therefore, more effective composition for prevention and treatment of a joint disorder is required.

Incidentally, the vitamin B1 has a problem of stability. Since the content of the vitamin B1 reduces during preservation owing to an environmental factor such as heat, pH, light and water, and also dosage forms and various coexisting components of the preparation. There is need for producing the preparation to take a stability of the vitamin B1 into consideration.

Particularly, in the case where vitamins are incorporated in the high proportion, there is known the method by incorporating a stabilizer such as an antioxidant to make the preparation stable. The method, however, is not preferred from a viewpoint of safety. Moreover, when improving stability by dosage form designs such as double layer tablets, laminated tablets, and coating techniques, there is a demerit that a producing process comes to complicate.

In order to stabilize vitamin B1 in a solid preparation, Japanese Patent Application Laid-open No. 271072/1993 (JP-5-271072A) discloses a vitamin preparation comprising a tocopherol succinate or a salt thereof, and a vitamin B1 or an ascorbic acid, in which at least one component of those is coated with a covering agent. Japanese Patent Application Laid-open No. 2000-247879 discloses a vitamin preparation comprising a tocopherol succinate or a salt thereof, a vitamin B1, and a specific basic inorganic compound. Japanese Patent Application Laid-open No. 268127/1997 (JP-9-268127A) discloses a solid preparation comprising a vitamin B1 derivative, starch, and dibasic calcium phosphate (calcium hydrogen phosphate).

In order to stabilize a vitamin in a liquid preparation, Japanese Patent Application Laid-open No. 255069/1993 (JP-5-255069A) discloses a vitamin preparation for intravenous injection which comprises at least one vitamin of 13 kinds of essential vitamins, and at least one ingredient selected from leucine, isoleucine, methionine and valine. Japanese Patent Application Laid-open No. 145056/1994 (JP-6-145056A) discloses a liquid preparation comprising a vitamin B, in which vitamin B6 is incorporated in a specific amount relative to vitamin B2. Further, Japanese Patent Application Laid-open No. 12458/1997 (JP-9-12458A) discloses a method by incorporating fat in the form of emulsifier etc into a liquid preparation comprising a vitamin B1.

While, chondroitin sulfate or a derivative thereof is a biopolymer which is distributed largely throughout cartilage tissues, and is effective for protection of corneal surface, sensorineural deafness (acoustic trauma deafness), chronic nephritis, neuralgia, arthralgia, arthritis, lumbago, articulat periarthritis (frozen shoulder), prevention of adhesion after celiotomly (abdominal operation), etc. Many preparations comprising chondroitin sodium sulfate are commercially available.

For example, Japanese Patent Application Laid-open No. 503197/1997 (JP-9-503197A) discloses a composition for treating connective tissues of human beings and animal, which comprises a glycosaminoglycan such as chondroitin, and an aminosugar such as glucosamine. However, the composition does not contain a vitamin B1. Japanese Patent Application Laid-open No. 2000-53569 discloses a composition comprising L-carnitine, glycosaminoglycan, and an excipient, which is suitable for preventing and treating of a joint disorder. However, the composition does not contain a vitamin B1. The literature also discloses a composition comprising L-carnitine, chondroitin sulfate, and glucosamine. Further, the literature describes that the composition is suitable for preventing or treating a joint disorder. Japanese Patent Application Laid-open No. 2000-139408 discloses a food (e.g., a tablet-type confectionery) comprising glucosamine or salts thereof, organic acid, flavor-improving agent such as fruit juice and sodium chloride, if necessary, sugars and excipients.

There is such subject that glycosaminoglycan such as chondroitin sulfate, which is a kind of polysaccharide, forms hydrogel cluster (massive hydrogel) in water. Particularly, if the concentration of glycosaminoglycan is high, hydrogel cluster is led to be formed easily. The formation of hydrogel cluster is liable to be accelerated under an environment at low pH particularly, and formed hydrogel cluster are hardly soluble and interrupts permeation of water thereinto. Therefore, if dosed preparations contain water in gastrointestinal tract to form hydrogel cluster, the hydrogel cluster prevents permeation of water into the solid preparations. As a result, disintegration time is delayed, and releasing of active ingredients from a solid preparation is inhibited. Accordingly, in dosage form designs of a solid preparation, there is need to taking disintegrativity into consideration. Further, it is known that an internal environment of gaster changes widely according to an internal or external factor such as individual differences and meals, and a gaster's pH of healthy body changes within the range of 1.2 to 6.8. It is needed to take into account in inhibiting a gelation of chondroitin sodium sulfate and in accelerating the disintegration under any internal environment.

### SUMMARY OF THE INVENTION

It is, therefore, an object of the present invention to provide a preparation in which a vitamin B1 can be effectively stabilized and a method of stabilizing the vitamin B1.

It is another object of the invention to provide a solid preparation capable of inhibiting formation of hydrogel cluster, and a method of improving disintegrativity of the solid preparation, even though the solid preparation comprises a glycosaminoglycan such as chondroitin sulfate.

A yet another object of the invention is to provide a method of inhibiting formation of hydrogel cluster of glycosaminoglycans and improving the disintegrativity of the solid preparation even though pH changes.

It is further object of the invention to provide a composition effective for preventing or treating a joint disorder such as arthralgia, arthritis, osteoarthritis, and joint stiffness.

It is still further object of the present invention to provide a composition useful for promoting joint flexibility and mobility (extension of mobile range (or mobile limit) of a joint) and maintaining comfortable joint movement.

The present inventors did much investigation to accomplish the above objects, and as a result, found that the use of aminosugars such as glucosamine makes a vitamin BI stable in a preparation so that the vitamin B1 can remain for a long term, and further formation of gel of glycosaminoglycans such as chondroitin sulfate is significantly inhibited and disintegration is accelerated in gastrointestinal tract so that active or effective ingredients can be stably released. Furthermore, the present inventors found that the preparation stabilized according to the present invention is effective for improving or palliating a joint disorder such as arthralgia and arthritis, and useful for promoting joint flexibility and mobility (extension of mobile range (or mobile limit) of a joint) and maintaining comfortable joint movement. The present invention has been accomplished based on the above findings.

That is, a preparation of the present invention comprises a vitamin B1 and an aminosugar (e.g., glucosamine) in an effective amount for stabilizing the vitamin B1, for example, not less than 0.1 part by weight relative to 1 part by weight of the vitamin B1. Moreover, the preparation of the present invention comprises a vitamin B1 and an aminosugar, in which the proportion of the vitamin B1 is 0.001 to 30% by weight based on the total amount of the preparation. The preparation of the present invention may comprise a composition for preventing or treating a joint disorder (e.g., arthralgia) composed of a vitamin B1 and an aminosugar (especially, glucosamine) in the above proportion. The preparation of the present invention may be a liquid, a solid preparation is advantageously available in the case that preparations comprise glycosaminoglycans liable to form gel because the aminosugar improves the disintegrativity (disintegration properties). That is, the preparation of the present invention may be a solid preparation comprising further a glycosaminoglycan (e.g., hyaluronic acid, chondroitin and salts thereof). The preparation of the present invention may be pharmaceutical preparation, a supplement or a confectionery.

The present invention also includes a method of stabilizing a vitamin B1, in which an aminosugar is incorporated to a preparation comprising the vitamin B1. Further, the present invention also includes a method of improving the disintegrativity, in which the aminosugar is incorporated to a preparation comprising a glycosaminoglycan.

Incidentally, in the specification, the term “preparation” is sometimes used in synonymously with the term “composition”.

### DETAILED DESCRIPTION OF THE INVENTION

A vitamin B1 contained in a preparation of the present invention includes thiamin, thiamin derivatives and salts thereof. Thiamin derivatives may be disulfide type, acyl type, etc. As thiamin derivatives, there are exemplified bisthiamin, thiamin disulflde (TDS), thiamin dicetyl sulfate, benfothiamin (BTMP), prosulthiamin (TPD), frusulthiamin (TTFD), bisbenthiamin (BTDS), cycothiamin (CCT), octotiamin (TATD), allithiamin, thiamin propyldisulfide, thiamin tetrahydrofurfuryldisulfide (TPFD), dicethiamin (DCET), bisbuthiamin, bisibuthiamin (DBT), thiamin monophosphate disulfide, thiamin pyrophosphate, thiamin ethyldisulfide, thiamin propyldisulfide and the like. As thiamin salts, there are exemplified physiologically acceptable salts, for example, hydrochloric acid salts and nitric acid salts such as thiamin hydrochloride, thiamin nitrate, bisthiamin nitrate, dicethiamin hydrochloride, fursultiamine hydrochloride. These vitamin B1 are used singly or in combination.

Of these vitamin B1, the preferred vitamin B1 includes, from a viewpoint of stability, thiamin, thiamin disulfide, benfothiamin, frusulthiamin, bisbenthiamin, dicethiamin, thiamin ethyldisulfide, thiamin propyldisulfide. Particularly, bisbenthiamin, frusulthiamin, and thiamin are preferable from viewpoints of stability and absorbency.

The content of the vitamin B1 can be selected within the range of, for example, about 0.001 to 30% by weight, preferably about 0.01 to 20% by weight, more preferably about 0.1 to 10% by weight, and is usually 0.1 to 5% by weight based on the total amount of the preparation (particularly, a solid preparation). In case of liquid preparations, the content of the vitamin B1 is preferably and generally about 0.0002 to 0.03 weight/volume % based on the whole preparation.

Incidentally, the vitamin B1 may be used in combination with other vitamins. As other vitamins, there are exemplified water-soluble vitamins [e.g., a vitamin B such as a vitamin B2 (riboflavins such as flavin adenine dinucleotide sodium, riboflavin, riboflavin sodium phosphate, riboflavin tetrabutyrate), a vitamin B6 (e.g., vitamin B6, pyridoxines such as pyridoxine and pyridoxal, physiologically acceptable salts (e.g., hydrochloric acid salts such as pyridoxine hydrochloride, acetic acid salts corresponding to the above such as pyridoxine acetate, phosphoric acid salts such as pyridoxal phosphate)), and a vitamin B12 (e.g., vitamin B12, cobalamins such as mecobalamin, cyanocobalamin, hydroxocobalamin, and methylcobalamin, or physiologically acceptable salts thereof (e.g., hydrochloric acid salts, acetic acid salts such as hydroxocobalamin acetate)); a vitamin C (e.g., ascorbic acid, calcium ascorbate, sodium ascorbate); a nicotinic acid (e.g., nicotinic acid, nicotinic acid amide); a pantothenic acid (e.g., panthenol, pantothenic acid or salts thereof); biotin; folic acid], fat-soluble vitamins [e.g., a vitamin A (retinol acetate, retinol palmitate, vitamin A oil), a vitamin D (e.g., ergocalciferol, cholecalciferol), a vitamin E (e.g., cod liver oil, enriched cod liver oil, d-α-tocopherol acetate, dl-α-tocopherol acetate, d-α-tocopherol, dl-α-tocopherol), a vitamin K]. The vitamins can be used singly or in combination. The vitamin B1 can be usually used in combination with the water-soluble vitamin (e.g., a vitamin B) such as the vitamin B6 and the vitamin B12.

The present invention has an advantage of incorporating a vitamin B6 and/or a vitamin B12. That is, it is known that the stability of the vitamin B6 or the vitamin B12 is reduced under the existence of a vitamin B1. Consequently, since a preparation is required to avoid contacting the components with each other by means of producing techniques such as double layer tablets, the producing process comes to be complicated. The present invention stabilizes the vitamin B1, and enables to incorporate the vitamin B6 and/or the vitamin B12 more stably. Incidentally, the preferred vitamin B6 is pyridoxine, and the preferred vitamin 12 is cyanocobalamin or hydroxocobalamin. Incidentally, it is preferred that the vitamin C, the vitamin B6 and/or the vitamin B12 are used in combination in order to improve or palliate arthralgia comprehensively and effectively.

The ratio (weight ratio) of the vitamin B1 and other vitamins may be selected within the range of the former/the latter=about 100/0 to 20/80, preferably about 100/0 to 30/70, and more preferably 100/0 to 50/50.

The vitamin B1 can be effectively stabilized in combination with the aminosugar. As aminosugars, there are exemplified sialic acid, muramic acid, glucosamines (e.g., glucosamine), salts thereof [e.g., glucosamine salts (e.g., physiologically acceptable salts such as hydrochloric acid salt and sulfuric acid salts, for example, inorganic acid salts such as glucosamine hydrochloride, glucosamine sulfate, glucosamine phosphate)], derivatives thereof [e.g., glucosamine derivatives (e.g., N-acetyl glucosamine, N-methyl-L-glucosamine). Amino sugars may be D-, L-, or DL-form. The aminosugars can be used singly or in combination. Preferred aminosugars are glucosamine or a salt thereof (e.g., glucosamine hydrochloric acid salt), N-acetyl glucosamine or a salt thereof.

Incidentally, glucosamine or a salt thereof typical of aminosugar can be obtained by treating crevettes, crab, calamary, etc by means of enzyme or hydrolysis, and purifying those, and also commercial products of glucosamine or a salt thereof can be used.

The content of aminosugars such as glucosamine can be selected within the wide range of about 1 to 99.9% by weight based on the total amount of the preparation (particularly, solid preparation), and is usually about 5 to 99.9% by weight (e.g., about 7.5 to 99.9% by weight), preferably about 10 to 90% by weight, more preferably about 10 to 80% by weight, and particularly about 10 to 60% by weight. The content of aminosugars in liquid preparation is, for example, about 0.001 to 10 w/v %, preferably about 0.01 to 10 w/v %, and more preferably about 0.01 to 5 w/v %.

The ratio of the aminosugar to the vitamin B1 may be an effective amount. For example, the proportion of the aminosugar can be selected within the range of not less than 0.1 part by weight (e.g., about 0.1 to 1000 parts by weight), preferably not less than 0.5 part by weight (e.g., about 1 to 500 parts by weight), more preferably not less than 1 part by weight (e.g., about 1 to 100 parts by weight), and particularly about 2 to 50 parts by weight relative to 1 part by weight of the vitamin B1.

The aminosugar can be functioned as a stabilizer to the vitamin B1, and incorporation of the aminosugar can make the vitamin B1 stable effectively. Therefore, the present invention also includes a method of stabilizing the vitamin B1 by incorporating the aminosugar to a preparation comprising the vitamin B1.

Further, according to the present invention, the combination of the vitamin B1 and the glucosamine can improve or palliate a joint disorder effectively, and is useful in a composition for preventing and treating a joint disorder. Moreover, since the incorporation of the glucosamine can make the vitamin B1 stable effectively, physiological activity or pharmacological activity of the vitamin B1 can be utilized effectively.

In particular, in a composition for preventing or treating a joint disorder constituting the preparation, it is sufficient that the proportion of the aminosugar (especially, glucosamine) is not less than effective amount for improving a joint disorder. For example, the proportion of the aminosugar (especially glucosamine) relative to 1 part by weight of the vitamin B1 is about 0.1 to 1000 parts by weight, preferably about 1 to 500 parts by weight, more preferably about 1 to 300 parts by weight, and is usually about 1 to 100 parts by weight, particularly about 2 to 50 parts by weight.

As described above, since the vitamin B1 can be stabilized by combination with the aminosugar, the vitamin B1 is available for various preparations comprising the vitamin B1 as an active ingredient (a physiologically active ingredient, a pharmacologically active ingredient), for example, liquid preparation. However, in the case that the vitamin B1 uses in combination with a glycosaminoglycan, a shape or form of a preparation comprising them is usually solid. That is, the aminosugar functions as disintegrator, and accelerates the disintegrativity of a solid preparation comprising the glycosaminoglycan. Therefore, the present invention includes not only a composition (or a solid preparation, or a composition for a solid preparation) comprising the aminosugar and the glycosaminoglycan regardless of the existence of the vitamin B1, also a method of improving disintegrativity of a solid preparation comprising the glycosaminoglycan by incorporating aminosugars such as glucosamine.

As described above, a solid preparation of the present invention may further comprise a glycosaminoglycan (mucopolysaccharide or acidic mucopolysaccharide). In particular, when the glycosaminoglycan is used, the physiological or pharmacological activity against a joint disorder can be further enhanced. The joint disorder includes, for example, arthralgia, arthritis, osteoarthritis, or stiffness. The glycosaminoglycan is used as an active ingredient (a physiologically active ingredient or a pharmacologically active ingredient) of the preparation. The glycosaminoglycan is a series of acidic polysaccharides (acidic glycans) including an aminosugar, for example, hyaluronic acid, chondroitin, jyaluronic acid, heparan, keratan sulfated glycosaminoglycan (glycosaminoglycan sulfate) [e.g., a chondroitin sulfate such as chondroitin sulfate A (chondroitin 4-sulfate), chondroitin sulfate B (dermatan sulfate), and chondroitin sulfate C (chondroitin 6-sulfate), heparin, heparan sulfate, keratan sulfate I, keratan sulfate II], or salts thereof, etc. As glycosaminoglycan salts or sulfated glycosaminoglycan salts, there are exemplified alkaline metal salts (e.g., sodium salts such as sodium hyaluronate, potassium salt), alkaline earth metal salts (e.g., calcium or magnesium salt), transition metal salt (e.g., iron or manganese salt), an organic salt (e.g., ammonium salts), etc. The glycosaminoglycans can be used singly or in combination.

Preferred glycosaminoglycan includes hyaluronic acid or salts thereof (e.g., sodium hyaluronate), chondroitin, chondroitin sulfate or salts thereof (e.g., metal salts of chondroitin sulfate). Particularly, chondroitin, chondroitin sulfate or salts thereof are preferable.

Chondroitin or salts thereof can be obtained from natural products such as cartilage and collagen of animal, and also commercial products of chondroitin or salts thereof can be used. Not only purified chondroitin also powders and extracts of an animal cartilage comprising chondroitin or salts thereof can be used. As salts, any physiologically acceptable salts such as hydrochloric acid salts and sulfuric acid salts are available. From viewpoints of safety and absorbency, purified chondroitin, or purified chondroitin sulfate or salts thereof are preferred.

The content of the glycosaminoglycan such as chondroitin sulfate can be selected within the wide range of about 0.5 to 90% by weight based on the total amount of the preparation, and is, for example, about 1 to 90% by weight, preferably about 5 to 80% by weight, more preferably about 10 to 70% by weight, usually about 10 to 60% by weight, and particularly about 10 to 50% by weight.

The proportion of the aminosugar relative to the glycosaminoglycan (e.g., chondroitin sulfate) is not particularly restricted, as far as the disintegrativity of the solid preparation is not deteriorated, and can be selected within the wide range of about 0.01 to 100 parts by weight. The proportion of the aminosugar is, for example, not less than 0.1 part by weight (e.g., about 0.1 to 50 parts by weight), preferably not less than 0.2 part by weight (e.g., about 0.2 to 30 parts by weight), and more preferably not less than 0.3 part by weight (particularly about 0.3 to 10 parts by weight, usually about 0.5 to 5 parts by weight).

When glucosamine is used as the aminosugar, the proportion of the glycosaminoglycan (e.g., chondroitin sulfate) can be selected within the wide range, for example, about 1 to 500 parts by weight relative to 100 parts by weight of the total amount of the vitamin B1 and the glucosamine. The proportion of the glucosamine is about 10 to 300 parts by weight (e.g., about 20 to 300 parts by weight), preferably about 30 to 200 parts by weight, and more preferably about 50 to 150 parts by weight relative to 100 parts by weight of the total amount of the vitamin B1 and the glucosamine.

The preparation of the present invention, if necessary, may comprise other physiologically active ingredients and pharmacologically active ingredients, for example, analgesic ingredients for a joint and muscular (e.g., analgesic and antipyretic agent and antiinflammatory agent such as acetaminophen, ibuprofen, salicylic acid derivatives, and mefenamic acid, antihistaminic agent), aminoethylsulphonic acid, γ-orizanol, crude drug ingredients (e.g., processed garlic, ginseng, coix seed), inorganic salts [e.g., monopottasium L-aspartate and monomagnesium di-L-asparate mixture, calcium glycerophosphate, calcium gluconate, precipitated calcium carbonate, calcium lactate, dibasic calcium phosphate anhydride (calcium hydrogenphosphate anhydride), dibasic calcium phosphate (calcium hydrogenphosphate)], caffeines (e.g., caffeine, anhydrous caffeine), amino acid or salts thereof (e.g., L-cysteine, L-cysteine hydrochloride), glucuronolactone, glucuronic acid, minerals.

Further, the dosage form of the preparation of the present invention is not particularly restricted, and may be liquid (liquid preparation) (e.g., suspension, emulsifier, syrup, injection solution) or a solid preparation (e.g., powder, fine subtilaes, granule, pill, capsule, tablet). The solid preparation also includes a pharmaceutical preparation, a supplement, an auxiliary health food, a confectionery (e.g., candy, oleaster, nougat). Incidentally, even though liquid comprises the glycosaminoglycan, the vitamin B1 can be stabilized. Therefore, liquid may comprise the glycosaminoglycan, but a solid preparation is preferred for utilizing a function of accelerating disintegrativity of aminosugars.

A preparation of the present invention can be prepared by a conventional manner such as adding a conventional carrier component, according to the dosage form of the preparation, as far as the stability etc is not deteriorated. As carrier components or additives in a solid preparation, there are exemplified excipients (e.g., sugar alcohols such as D-sorbitol, D-mannitol, and xylitol, sugars such as glucose, sucrose, lactose, and fructose, crystalline cellulose, carmellose sodium, dibasic calcium phosphate, wheat starch, rice starch, corn starch, potato starch, dextrin, β-cyclodextrin, light silicic anhydride, titanium oxide, magnesium aluminometasilicate, talc, kaolin); disintegrators (e.g., low-substituted hydroxypropylcellulose, carboxymethylcellulose calcium, croscarmellose sodium, hydroxypropylstarch, partially alpha-starch); binders (e.g., cellulose derivatives such as methyl cellulose, ethyl cellulose, hydroxypropylcellulose, and hydroxypropylmethylcellulose, polyvinylpyrrolidone, polyvinylalcohol, acrylic polymer, gelatin, acacia, pullulan, alpha-starch, agar, tragacanth, sodium alginate, algiginic acid propylene glycol ester (propylene glycol alginate)); lubricants (stearic acid, magnesium stearate, calcium stearate, polyoxyl stearate, cetanol, talc, hardened oil, sucrose ester of fatty acid, dimethylpolysiloxane, yellow beeswax, white beeswax); antioxidants (e.g., dibutylhydroxytoluene (BHT), propyl gallate, butylhydroxyanisole (BHA), tocopherol, citric acid); coating agent (e.g., hydroxypropylmethylcellulose, hydroxypropylcellulose, methylcellulose, ethylcellulose, hydroxypropylmethylcellulose phthalate, hydroxypropylmethylcellulose acetate succinate, carboxymethylethylcellulose, cellulose acetate phthalate, polyvinyl acetal diethylaminoacetate, aminoalkyl(meth)acrylate copolymer, (meth)acrylic acid copolymer, polyvinyl acetal diethylaminoacetate, shellac); colorants (e.g., extract of turmeric, riboflavin, titanium oxide, carotene liquid); corrigents (e.g., aspartame, ascorbic acid, Stevia rebaudiana, menthol, crude glycyrrhiza extract, simple syrup); surfactants (e.g., polyoxyethylene-hardened castor oil, glyceryl monosterate, sorbitan monosterate, sorbitan monolaurate, polyoxyethylenepolyoxypropylene, polysorbates, sodium lauryl sulfate, macrogols, sucrose ester of fatty acid); plasticizers (e.g., triethyl citrate, polyethylene glycol, triacetin, cetanol); edulcorants (e.g., natural or synthetic edulcorants such as sucrose, mannitol, and aspartame); aromatizing agents (e.g., menthol); absorbents; preservatives; wetting agents; antistatic agents.

As carrier component in liquid preparation, water or alcohol-containing water can be usually employed, and a liquid preparation can be prepared with the use of a conventional component. As additives in liquid or solution, there are exemplified, for example, pH regulators (e.g., citric acid, malic acid, disodium hydrogenphosphate, dipotassium phosphate), refrigerants (e.g., 1-menthol, mentha water), the above surfactants, suspensions (e.g., kaolin, carmellose sodium, xanthan gum, methylcellulose, tragacanth), antifoamers (e.g., dimethylpolysiloxane, silicone antiformer), thickening agents (e.g., xanthan gum, tragacanth, methylcellulose, dextrin), solubilizers (e.g., ethanol, sucrose ester of fatty acid, macrogols), the above antioxidants, colorants, edulcorants, aromatizing agents.

The preparation of the present invention can be obtained by conventional manners of the concerned art field with or without modification suitably. For example, the tablets can be prepared by compression-molding which comprises mixing a powdered active ingredient and a pharmaceutically acceptable carrier component (e.g., excipient) and compressing, and confectionery tablets such as candy may be prepared by a method of injection into mold. Further, of solid preparations, powdered granules such as granule may be prepared by various granulations (e.g., extruding granulation, milling granulation, dry compacting granulation, fluidizing granulation, tumbling granulation, high-shear stirring granulation). The tablets can be prepared by using the granulation, molding (wet molding, direct molding), etc in combination suitably. Furthermore, capsules can be prepared by conventional manners in which powdered granule (e.g., powder, granule) is filled in capsule (soft or hard capsule). The preferred dosage form of the preparation of the present invention is a tablet (e.g., chewable type tablets). The tablet may be a sugar-coated tablet obtained by a sugar-coating. Further the tablets may be a single layer tablet or a laminated tablet such as a double layer tablet.

Liquid preparations can be prepared by dissolving or dispersing each ingredient in aqueous medium (e.g., purified water, ethanol-containing purified water) of a carrier component, if necessary, filtrating or sterilizing, filling up in the predetermined container and sterilizing.

Since the present invention can stabilize vitamin B1 by aminosugars such as glucosamine, the present invention is applied for a preparation comprising vitamin B1. Further, the present invention can improve the disintegrativity by inhibiting the gel formation of glycosaminoglycans such as chondroitin sulfate with the use of aminosugars. Therefore, the present invention is advantageously applied for a solid preparation comprising glycosaminoglycans. Incidentally, the solid preparation of the present invention is effective for accelerating the disintegration of the solid preparation in the digestive organs and stably releasing active ingredients with no influence of pH change. For example, even when pH changes within the range of about 1 to 10 (e.g., about 1 to 7), the solid preparation disintegrates effectively. Particularly, the disintegrativity in the range of low pH (e.g., about 1 to 4) can be significantly improved. Therefore, even when pH of inner gaster changes within the range of 1.2 to 6.8, the use for a preparation is applicable.

The preparation of the present invention is useful in mammals (e.g., humans, monkeys, sheep, bovines, horses, dogs, cats, rabbits, rats, mice, etc.) for the treatment a joint disorder (e.g., arthralgia). Thus, the present invention includes the method of treating or preventing a joint disorder (e.g., arthralgia) in a mammal which comprises administering a vitamin B1 and an aminosugar (e.g., effective amount of a pharmaceutical preparation composed of a vitamin B1 and an aminosugar in the specific proportion) to a subject. In this method, it is sufficient that effective amount of a vitamin B1 and an aminosugar is administered to a subject, and a preparation composed of a vitamin B1 and an aminosugar may be administered or a preparation composed of a vitamin B1 and a preparation composed of an aminosugar may be administered. In particular, the preparation (especially, pharmaceutical preparation) of the present invention is suitable for an oral administration, and can be administered one or plural times per day. The dose of the preparation per day for an adult is, for example, about 1 to 300 mg, preferably about 5 to 150 mg, more preferably about 5 to 100 mg, and particularly about 5 to 30 mg as a free vitamin B1. When vitamin B6 is incorporated, the dose of vitamin B6 per day for an adult is, in terms of a free vitamin B6, for example, about 1 to 300 mg, and preferably about 10 to 100 mg. Moreover, the dose of vitamin B12 per day for an adult is, in terms of a free vitamin B6, for example, about 10 to 3000 μg, and preferably about 50 to 1500 μg.

The dose of the aminosugar such as glucosamine per day for an adult is, in terms of a free aminosugar, for example, about 50 to 3000 mg, preferably about 100 to 2500 mg, more preferably about 300 to 2000 mg, and particularly about 500 to 1500 mg.

Further, the dose of the glycosaminoglycan such as chondroitin per day for an adult (as a free glycosaminoglycan) is, for example, about 0.01 to 10 g, preferably about 0.01 to 5 g, more preferably about 0.05 to 2 g, particularly about 0.1 to 1.7 g, and more particularly about 0.1 to 1.5 g.

The preparation of the present invention is useful as a pharmaceutical preparation (e.g., a pharmaceutical preparation effective for preventing and treating a joint disorder such as arthralgia and arthritis, and treating myalgia, with utilizing activities of the vitamin B1 and the chondroitin), and also can be used as foods (e.g., confectionery tablets, supplements or auxiliary health foods).

In the present invention, an aminosugar can make a vitamin B1 stable effectively. Moreover, even in a solid preparation comprising glycosaminoglycans such as chondroitin sulfate, a formation of hydrogel masses can be inhibited and the disintegrativity can be improved. Particularly, the dependency of disintegration on pH is low, and even when pH changes, the disintegrativity of a solid preparation can be improved effectively. Since the vitamin B1 is combined with the aminosugar (especially, the glucosamine), the solid preparation comprising them is useful for prophylaxis or therapy of arthralgia. Moreover, the mobile range of a joint can be effectively extended as well as an improvement of arthralgia. These effects come to be more effective by further comprising glycosaminoglycans.

### EXAMPLES

The following examples, comparative examples and experimental examples are merely intended to illustrate the present invention in further detail and should not be construed as defining the scope of the invention.

### Example 1

Into the mixed powder of thiamin nitrate, glucosamine hydrochloride, and crystalline cellulose which was mixed homogeneously, hydroxypropylcellulose dissolved in purified water was added, and the mixture was granulated with stirring. To powdered granule which was dried and sized were mixed monopottasium L-aspartate and monomagnesium di-L-asparate mixture, chondroitin sodium sulfate, croscarmellose sodium, light silicic anhydride, and magnesium stearate, and stirred the mixture homogeneously. The mixture was molded by a rotary tablet machine to obtain a circle tablet (8.5 mm in diameter, 270 mg in weight, 5 kg in hardness (measured by digital hardness tester)). Formulation of the tablet is described as follows. Incidentally, the term “parts” means “parts by weight”. Hereinafter, the term “parts” has the same meaning.

| | content |
|---|---|
| | (parts) |
| thiamin nitrate | 1.25 |
| monopottasium L-aspartate and monomagnesium di-L- | 8.3 |
| asparate mixture | |
| chondroitin sodium sulfate | 33.3 |
| glucosamine hydrochloride | 41.7 |
| hydroxypropylcellulose | 1.62 |
| crystalline cellulose | 5.33 |
| magnesium stearate | 1.5 |
| croscarmellose sodium | 6.0 |
| light silicic anhydride | 1.0 |
| total | 100 |

### Example 2

The granule (a dosage or bag=1500 mg) was produced with the use of the following formulation in accordance with the item “granules” in the general rule of preparation of the Pharmacopoeia of Japan.

| | Content |
|---|---|
| | (parts) |
| thiamin hydrochlorde | 0.6 |
| chondroitin sodium sulfate | 17.8 |
| glucosamine hydrochloride | 22.2 |
| riboflavin tetrabutyrate | 0.3 |
| pyridoxine hydrochloride | 0.3 |
| hydroxypropylcellulose | 2.4 |
| crystalline cellulose | 24.4 |
| mannitol | 31.8 |
| menthol | 0.2 |
| total | 100 |

### Example 3

### A Preparation Comprising Vitamin B1, Vitamin B6 and Vitamin B12

The tablet (a tablet=280 mg) was produced with the use of the following formulation in accordance with the item “tablets” in the general rule of the Pharmacopoeia of Japan.

| | Content |
|---|---|
| | (parts) |
| fursultiamine hydrochloride | 4.0 |
| chondroitin sodium sulfate | 23.8 |
| glucosamine hydrochloride | 35.7 |
| pyridoxine hydrochloride | 4.0 |
| hydroxocobalamin | 0.06 |
| hydroxypropylcellulose | 0.7 |
| crystalline cellulose | 31.24 |
| magnesium stearate | 0.5 |
| total | 100 |

### Example 4

### A Preparation Comprising Vitamin B1 and Vitamin E

The tablet (a tablet=270 mg) was produced with the use of the following formulation in accordance with the item “tablets” in the general rule of the Pharmacopoeia of Japan.

| | Content |
|---|---|
| | (parts) |
| thiamin nitrate | 1.2 |
| chondroitin sodium sulfate | 20.6 |
| glucosamine hydrochloride | 20.6 |
| d-α-tocopherol acetate | 0.5 |
| hydroxypropylcellulose | 3.0 |
| crystalline cellulose | 28.8 |
| mannitol | 24.7 |
| aromatizing agent | 0.1 |
| magnesium stearate | 0.5 |
| total weight | 100 |

### Example 5

### A Preparation Comprising Vitamin B1 and Hyaluronic Acid

The tablet (a tablet=500 mg) was produced with the use of the following formulation in accordance with the item “tablets” in the general rule of the Pharmacopoeia of Japan.

| | Content |
|---|---|
| | (parts) |
| thiamin hydrochloride | 0.8 |
| hyaluronic acid | 15 |
| glucosamine hydrochloride | 30 |
| hydroxypropylcellulose | 3 |
| crystalline cellulose | 17.3 |
| lactose | 33.5 |
| magnesium stearate | 0.5 |
| total weight | 100 |

### Example 6

The tablet (a tablet=350 mg) was produced with the use of the following formulation in accordance with the item “tablets” in the general rule of the Pharmacopoeia of Japan.

| | Content |
|---|---|
| | (parts) |
| thiamin nitrate | 0.1 |
| chondroitin sodium sulfate | 19.0 |
| glucosamine sulfate | 9.5 |
| hydroxypropylcellulose | 2.0 |
| mannitol | 68.9 |
| magnesium stearate | 0.5 |
| total weight | 100 |

### Example 7

### A Preparation Comprising Vitamin B1 and Vitamin C

The chewable tablet (a tablet=1300 mg) was produced with the use of the following formulation in accordance with the item “tablets” in the general rule of of the Pharmacopoeia of Japan.

| | content |
|---|---|
| | (parts) |
| thiamin nitrate | 0.8 |
| chondroitin sodium sulfate | 20.5 |
| glucosamine hydrochloride | 38.5 |
| ascorbic acid | 6.4 |
| hydroxypropylcellulose | 2.0 |
| mannitol | 30.7 |
| aspartame | 0.4 |
| menthol | 0.2 |
| magnesium stearate | 0.5 |
| total weight | 100 |

### Example 8

### A Preparation Comprising Vitamin B1 and Hyaluronic Acid

The tablet (a tablet=500 mg) was produced with the use of the following formulation in accordance with the item “tablets” in the general rule of the Pharmacopoeia of Japan.

| | Content |
|---|---|
| | (parts) |
| thiamin hydrochloride | 0.8 |
| chondroitin sulfate | 20 |
| N-acetylglucosamine | 30 |
| hydroxypropylcellulose | 3 |
| crystalline cellulose | 17.3 |
| lactose | 28.5 |
| magnesium stearate | 0.5 |
| total weight | 100 |

### Experimental Example 1

A mixture of a tablet component was prepared by mixing 50 parts by weight of chondroitin sodium sulfate (Seikagaku Kogyo Co., Ltd.,), 50 parts by weight of glucosamine hydrochloride (Yaidzu Suisan Co., Ltd.,), and 0.5 part by weight of magnesium stearate (Taihei Kagaku Co., Ltd.,) homogeneously. The mixture was molded by a rotary tablet machine to obtain a circle tablet similar to the tablet of Example 1 (8.5 mm in diameter, 270 mg in weight, 5 kg in hardness (measured by digital hardness tester)).

In contrast, as a comparative example, the tablets of comparative example 1 (crystalline cellulose) and comparative example 2 (lactose) were obtained by the same procedure as described above except for using 50 parts by weight of crystalline cellulose (Asahi Kasei Kogyo Co., Ltd.,) or 50 parts by weight of lactose (DMV Co., Ltd.,) instead of 50 parts by weight of glucosamine hydrochloride.

The disintegrativity of the obtained tablet to test solution was examined in accordance with the disintegration test (general test, 13th revision of the Pharmacopoea of Japan). Incidentally, a dosed solid preparation needs to be dissolved or dispersed in small particles for quick disintegration and elution in gaster.

As a test solution, the test solution having pH 1.2 (corresponding to the 1st solution of the disintegration test of the Pharmacopoeia of Japan) which was regulated by sodium chloride and hydrochloric acid, the test solution having pH 4.5 which was regulated by acetic acid buffer solution, the test solution having pH 6.8 (corresponding to the 2nd solution of the disintegration test of the Pharmacopoeia of Japan) which was regulated by monobasic potassium phosphate and sodium hydroxide on the assumption of pH in gaster of healthy body were prepared.

Each of tablets and each of test solutions were put to a glass test container, and the container was moved up and down smoothly for 29 to 32 times of both ways per minute, and 53 to 57 mm of amplitude as the temperature of the test solution was kept at 37° C. plus or minus 2° C. The time taken to reach the disappearance of the tablet was measured.

As the pH was lowered, the disintegration time of each tablet tends to become longer. The disintegrativity of Example 1 comprising glucosamine, however, is high at any given pH value. In comparative examples 1 and 2, the disintegration took long at particularly low pH, and formation of hydrogel masses of chondroitin sodium sulfate can not be inhibited. The results are shown in Table 1.

TABLE 1

| | | | Comparative | |
|---|---|---|---|---|
| | | Experimental | Example 1 | Comparative |
| | | Example 1 | (crystalline | Example 2 |
| | | (glucosamine) | cellulose) | (lactose) |
| Disintegration | pH = 1.2 | 19.5 | 27.5 | 29.5 |
| time | pH = 4.5 | 14.5 | 18.5 | 22.5 |
| (minute) | pH = 6.8 | 12.5 | 10.5 | 15.5 |
| | mean | 15.5 | 18.8 | 22.5 |

| | | | |
|---|---|---|---|
| Change coefficient of | 23.3% | 45.2% | 31.1% |
| disintegration time | | | |

### Experimental Example 2

A mixture of tablet components was prepared by mixing of 3 parts by weight of thiamin nitrate (Takeda Yakuhin Kogyo Co., Ltd.,), 100 parts by weight of glucosamine hydrochloride (Yaidzu Suisan Co., Ltd.,), and 0.5 part by weight of magnesium stearate (Taihei Kagaku Co., Ltd.,) homogeneously. The mixture was molded by a rotary tablet machine to obtain a circle tablet similar to the tablet of Example 2 (8.5 mm in diameter, 270 mg in weight, 5 kg in hardness (measured by digital hardness tester)).

Tablets of Example 2 and Comparative Example 2 were put into a glass bottle, and preserved at 50° C. under prevention of light-transmittance for 2 weeks. The residual thiamin nitrate in tablets was determined by high performance liquid chromatography (HPLC) in accordance with usual method, and, as a result, the residual ratio in Example 2 (the residual amount of thiamin nitrate in tablet/the initial amount of thiamin nitrate in tablet) is 99.8% while the residual ratio in Comparative Example 3 is 95.3%.

Moreover, tablet of Example 2 was put into a glass bottle, and preserved at 40° C. under prevention of light-transmittance for 1, 3, and 6 months, respectively. The residual ratio of thiamin nitrate in tablets was measured same as above, and, as a result, the each residual amount after 1 and 3 months later was 100%, and the residual amount after 6 months later is 99.9%, and the high ratio was maintained. Therefore, the results indicated that glucosamine is effective for stabilizing thiamin nitrate for a long term.

### Experimental Example 3

In the test tablets 1 to 2 and the preparations 9 and 10 shown in Table 2, effect of improvement for arthralgia of 10 persons who have a pain of a joint genus due to transformation of a joint genus was evaluated. Incidentally, the test tablets are prepared in accordance with formulation of Example 1 except for changing the amount of active ingredients.

The evaluation test was carried out by comparing the degree of arthralgia and joint stiffness between before and after administered in such test condition that 3 pieces of tablets were administered each time 3 times per day for 6 weeks. The evaluation was made by the degree of pain when walking, going up and down the stairs, standing up from the chair, and sitting properly (sitting erect with one's legs folded under one, Japanese fashion), and each case was marked. The marking of the degree of arthralgia and joint stiffness was performed according to the following criteria.

[Pain When Walking]

no pain: 0

feel pain when walking for long distance: 1

feel pain even when walking for short distance: 2

[Pain When Going Up and Down the Stairs]

go up and down easily: 0

possible to go up and down with the use of handrail: 1

possible to go up and down step by step: 2

[Pain When Standing Up From the Chair]

no pain: 0

feel pain unless supporting oneself with hand: 1

feel pain even if supporting oneself with hand: 2

[Pain When Sitting Properly]

possible to sit properly: 0

impossible to sit properly and possible sitting informally (with one's legs doubled back to one side): 1

impossible to sit informally: 2

As to each item, the effect was evaluated by the difference of the total marks of 10 persons between before and after administered. That is, the larger the difference of the total marks Δ is, the higher the effect of improvement is. The results are shown in Table 2.

Moreover, the degree of arthralgia on whole daily life was evaluated by VAS (Visual Analogue Scale). That is, as a evaluation method, when it was assumed that “no pain” marks 0 at the left end of a scale (10 cm) and “most highest pain imaginable” marks 100 at right end of the scale. The position on the scale where the degree of subjective pains was represented was pointed out by a subject, and a pointed mark on scale was examined. The effect was evaluated by the difference of the total marks of 10 persons between before and after administered.

TABLE 2

| | Example | Example | Test | Test |
|---|---|---|---|---|
| | 9 | 10 | tablet 1 | tablet 2 |
| thiamin nitrate | 30 mg | 10 mg | — | 30 mg |
| chondroitin sodium | 800 mg | 800 mg | 800 mg | 800 mg |
| sulfate | | | | |
| glucosamine | 1000 mg | 1000 mg | 1000 mg | — |
| hydrochloride | | | | |

TABLE 3

| | | | Test | Test |
|---|---|---|---|---|
| | Example | Example | table | Tablet |
| | 9 | 10 | t 1 | 2 |
| Walking | Δ8 | Δ7 | Δ4 | Δ3 |
| Going up and down | Δ5 | Δ5 | Δ4 | Δ2 |
| the stairs | | | | |
| Standing up from | Δ7 | Δ6 | Δ4 | Δ3 |
| the chair | | | | |
| sitting properly | Δ5 | Δ6 | Δ3 | Δ2 |
| total of 4 items | Δ25 | Δ24 | Δ15 | Δ10 |
| VAS | Δ223 | Δ217 | Δ166 | Δ129 |

As apparent from the above Tables demonstrating, examination results concerning the effect of improvement with the degree of arthralgia and stiffness on 4 items of daily life movements of walking, going up and down the stairs, standing up from the chair, and sitting properly, and with VAS, arthralgia was improved in any movements by a preparation of the present invention further comprising vitamin B1, and excellent effects were admitted as compared with a preparation comprising chondroitin sodium sulfate and glucosamine hydrochloride,. Moreover, the improvement of arthralgia and joint stiffness on daily life movement indicates Lightening a restriction of mobile range of a joint, and the present invention can extend mobile range for a joint, and the present invention has an effect on promoting and maintaining normal joint flexibility and mobility.

Further, as compared with a preparation comprising vitamin B1 and chondroitin sodium sulfate, the preparation of the present invention comprising further glucosamine has an excellent effect.

Accordingly, improvement of arthralgia and stiffness also suggests improvement of arthritis, and thus, the composition of the present invention is effective for treating a joint disorder.

What is claimed is:

1. A preparation comprising a vitamin B1, which comprises an aminosugar in the proportion of not less than 0.1 part by weight relative to 1 part by weight of the vitamin B1.

2. A preparation according to claim 1, which comprises a composition for treating or preventing a joint disorder composed of a vitamin B1 and an aminosugar.

3. A preparation according to claim 2, wherein the joint disorder includes arthralgia, arthritis, osteoarthritis, or stiffness.

4. A preparation according to claim 1, which comprises the vitamin B1 and the aminosugar, wherein the proportion of the vitamin B1 is 0.001 to 30% by weight based on the total amount of the preparation.

5. A preparation according to claim 1, wherein the aminosugar comprises a glucosamine.

6. A preparation according to claim 5, wherein the glucosamine comprises glucosamine or a salt thereof.

7. A preparation according to claim 5, which comprises a composition for treating or preventing arthralgia composed of a vitamin B1 and a glucosamine wherein the proportion of the glucosamine is 0.1 to 1000 parts by weight relative to 1 part by weight of the vitamin B1.

8. A preparation according to claim 1, which is a solid preparation further comprising a glycosaminoglycan.

9. A preparation according to claim 8, wherein the glycosaminoglycan comprises at least one member selected from a hyaluronic acid, a chondroitin and a salt thereof.

10. A preparation according to claim 8, wherein the proportion of the aminosugar is not less than 0.1 part by weight relative to 1 part by weight of the glycosaminoglycan.

11. A preparation according to claim 8, wherein the proportion of the glycosaminoglycan is 10 to 300 parts by weight based on 100 parts by weight of the total amount of the vitamin B1 and the glucosamine.

12. A preparation according to claim 8, wherein the proportion of the glucosamine is 10 to 500 parts by weight relative to 1 part by weight of the vitamin B1, and the proportion of the glycosaminoglycan is 30 to 200 parts by weight relative to 100 parts by weight of the total amount of the vitamin B1 and the glucosamine.

13. A preparation according to claim 1, which comprises vitamin B1, an aminosugar and at least one ingredient selected from the vitamin B1 and the vitamin B12.

14. A preparation according to claim 13, wherein the ratio of the vitamin 1 to the ingredient is 100/0 to 20/80.

15. A preparation according to claim 1, which is pharmaceutical preparation, a supplement, an auxiliary health food or a confectionery.

16. A method of stabilizing a vitamin B1, which comprises incorporating an aminosugar into a preparation comprising Vitamin B1.

17. A method of improving a disintegrativity of a solid preparation, which comprises incorporating an aminosugar into the preparation comprising a glycosaminoglycan.

18. A method of treating and preventing a joint disorder in a mammal which comprises administering a vitamin B1 and an aminosugar in proportion of the aminosugar of not less than 0.1 part by weight relative to 1 part by weight of the vitamin B1 to a subject.

19. A method according to claim 18, which comprises administering a preparation composed of a vitamin B1 and an aminosugar, or administering a preparation composed of vitamin B1 and a preparation composed of an aminosugar.

20. A method of treating and preventing a joint disorder in a mammal which comprises administering effective amount of a pharmaceutical preparation to a subject, wherein the preparation comprises a vitamin B1 and an aminosugar in proportion of the aminosugar of not less than 0.1 part by weight relative to 1 part of by weight of the vitamin B1.

21. A method according to any one of claims 16, 17, 18 and 20, wherein the aminosugar is glucosamine.
